(19) United States
(12) Patent Application Publication
(10) Pub. No.: US 2025/0100969 A1
(43) Pub. Date: Mar. 27, 2025

(54) COMPOUND, RUBBER BLEND CONTAINING THE COMPOUND, VEHICLE TIRE COMPRISING THE RUBBER BLEND IN AT LEAST ONE COMPONENT, PROCESS FOR PRODUCING THE COMPOUND, AND USE OF THE COMPOUND AS AN AGE RESISTOR AND/OR ANTIOXIDANT

(71) Applicant: Continental Reifen Deutschland GmbH, Hannover (DE)

(72) Inventors: Andreas Jacob, Hannover (DE); David-Raphael Dauer, Dassel (DE); Julian Strohmeier, Einbeck (DE); Jörg-August Becker, Hannover (DE); Florian Matz, Hannover (DE)

(73) Assignee: Continental Reifen Deutschland GmbH, Hannover (DE)

(21) Appl. No.: 18/714,888

(22) PCT Filed: Nov. 2, 2022
(86) PCT No.: PCT/DE2022/200254

§ 371 Date: May 30, 2024

(30) Foreign Application Priority Data
Dec. 2, 2021 (DE) 10 2021 213 720.2

(51) Int. Cl. C07D 209/88; B60C 1/00; C08K 3/04; C08K 5/3417

(52) U.S. Cl. CPC C07D 209/88; B60C 1/0016; B60C 1/0025; C08K 3/04; C08K 5/3417; C08K 2201/019

(57) ABSTRACT

A compound, to a rubber mixture containing the compound, to a vehicle tire comprising the rubber mixture in at least one component, to a process for producing the compound and to the use of the compound as an aging stabilizer and/or antioxidant. The compound has the following formula I):

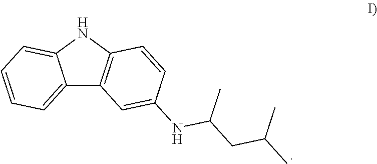 [SMILES: CC(C)CC(C)Nc1ccc2[nH]c3ccccc3c2c1]

This application is a National Stage Application under 35 U.S.C. § 371 of International Patent Application No. PCT/DE2022/200254 filed on Nov. 2, 2022, which claims priority to German Application No. 10 2021 213 720.2 filed Dec. 2, 2021, the disclosures of which are herein incorporated by reference in their entireties.

The invention relates to a compound, to a rubber mixture containing the compound, to a vehicle tire comprising the rubber mixture in at least one component, to a process for producing the compound and to the use of the compound as an aging stabilizer and/or antioxidant.

It is known that vehicle tires and technical rubber articles employ polymeric materials such as especially rubbers.

In case of prolonged storage and especially in the target application which is often at elevated temperatures, natural rubber and synthetic polymers (such as IR, BR, SSBR, ESBR etc.) but also natural and synthetic oils, fats and lubricants are subject to oxidation reactions which have an adverse effect on the originally desired properties. Depending on the type of the polymer the polymer chains are shortened right up to the liquefaction of the material or subsequent hardening of the material occurs.

Aging stabilizers thus play a decisive role in the durability of vehicle tires and other technical rubber articles.

Known aging stabilizers are aromatic amines, for example 6PPD (N-(1,3-dimethylbutyl)-N′-phenyl-p-phenylenediamine), IPPD (N-Isopropyl-N′-phenyl-p-phenylenediamine) or SPPD (N-(1-phenylethyl)-N′-phenyl-p-phenylenediamine).

These molecules can react with oxygen or ozone or free radicals formed, such as alkyl, alkoxy and alkylperoxy radicals, and thus scavenge these and accordingly protect the rubbers etc. from further oxidation reactions.

However, a disadvantage of this substance class is that they are suspected to be carcinogenic.

Aging stabilizers which especially react with ozone and effect scavenging thereof are also referred to as “antiozonants”.

It is an object of the invention to provide a novel compound which can especially be used as an aging stabilizer in vehicle tires or other technical rubber articles, specifically with a lower hazard potential coupled with sufficient solubility in the respective matrix, for example and in particular in the polymer. This is intended to ensure continuing optimal protection from oxygen and ozone and to prevent the tendency for blooming while reducing hazardousness to health.

The object is achieved by the inventive compound having formula (I), by the inventive rubber mixture containing the compound and also by the inventive vehicle tire comprising the inventive rubber mixture in at least one component. The object is further achieved by the processes for producing the compound and also the use of the compound as an aging stabilizer and/or antioxidant and/or antiozonant.

The compound has the formula I):

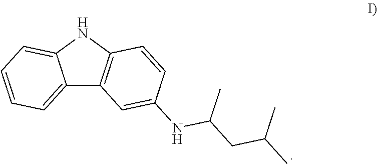 [SMILES: CC(C)CC(C)Nc1ccc2[nH]c3ccccc3c2c1]

The compound of formula I) is thus N-(1,3-dimethylbutyl)-9H-carbazol-3-amine or else, according to an alternative designation, 3-(1,3-dimethylbutylamino)-9H-carbazole.

The compound according to the invention shows an aging stabilization effect similar to 6PPD in rubber mixtures and is therefore suitable as a substitute for 6PPD, whose decomposition products are extremely toxic to silver salmon and thus presumably also to other aquatic organisms.

The compound according to the invention surprisingly also exhibits very good solubility in rubber mixtures, in particular for vehicle tires and other technical rubber articles, which could be attributable to the 1,3-dimethylbutyl group on the nitrogen atom. However, the invention is not to be bound to any particular mechanism of action or any particular theory.

The invention comprises all advantageous embodiments which are reflected inter alia in the claims. The invention especially also comprises embodiments which result from a combination of different features with different levels of preference for these features so that the invention also comprises a combination of a first feature described as “preferred” or described in the context of an advantageous embodiment with a further feature described for example as “particularly preferred”.

The inventive compound according to formula I) is particularly suitable as an aging stabilizer and/or antiozonant in vehicle tires and/or other technical rubber articles, such as in particular an air spring, bellows, conveyor belt, belt, drive belt, hose, rubber band, profile, a seal, a membrane, tactile sensors for medical applications or robotics applications, or a shoe sole or parts thereof and/or oils and/or lubricants.

The inventive compound according to formula I) is particularly suitable for producing a rubber article, in particular an air spring, bellows, conveyor belt, belt, drive belt, hose, rubber band, profile, a seal, a membrane, tactile sensors for medical applications or robotics applications, or a shoe sole or parts thereof.

To use the compound of formula I) in the recited articles or substances, said compound is used in a composition and employed incorporated in said composition.

In vehicle tires or other technical rubber articles said composition is in particular a rubber mixture.

The present invention further provides for the use of the inventive compound of formula I) in oils, lubricants, such as in particular fuels or fluids for engines. The compound according to the invention may thus especially be used in engines.

As mentioned above the invention further provides a rubber mixture.

The rubber mixture according to the invention contains the compound of formula I). The rubber mixture according to the invention may in principle be any rubber mixture in which in particular the novel inventive compound of formula I) acts as an aging stabilizer and/or antiozonant at low toxicity.

The rubber mixture of the invention contains at least one rubber.

It is preferable when the rubber mixture according to the invention contains 0.1 to 10 phr, particularly preferably 0.1 to 6 phr, very particularly preferably 1 to 5 phr, of the compound of formula I).

The unit “phr” (parts per hundred parts of rubber by weight) used in this document is the conventional indication of quantity for mixture recipes in the rubber industry. The dosage of the parts by weight of the individual substances is based in this document on 100 parts by weight of the total mass of all high molecular weight (M_{w} greater than 20 000 g/mol) rubbers present in the mixture.

In advantageous embodiments of the invention the rubber mixture according to the invention contains at least one diene rubber.

The rubber mixture may accordingly contain a diene rubber or a mixture of two or more different diene rubbers.

Diene rubbers are rubbers which are formed by polymerization or copolymerization of dienes and/or cycloalkenes and thus have C═C double bonds either in the main chain or in the side groups.

The diene rubber is preferably selected from the group consisting of natural polyisoprene (NR), synthetic polyisoprene (IR), epoxidized polyisoprene (ENR), butadiene rubber (BR), butadiene-isoprene rubber, solution-polymerized styrene-butadiene rubber (SSBR), emulsion-polymerized styrene-butadiene rubber (ESBR), styrene-isoprene rubber, liquid rubbers having a molecular weight M_{w} of more than 20 000 g/mol, halobutyl rubber, polynorbornene, isoprene-isobutylene copolymer, ethylene-propylene-diene rubber, nitrile rubber, chloroprene rubber, acrylate rubber, fluororubber, silicone rubber, polysulfide rubber, epichlorohydrin rubber, styrene-isoprene-butadiene terpolymer, hydrogenated acrylonitrile butadiene rubber and hydrogenated styrene-butadiene rubber.

Nitrile rubber, hydrogenated acrylonitrile-butadiene rubber, chloroprene rubber, butyl rubber, halobutyl rubber and/or ethylene-propylene-diene rubber in particular are used in the production of technical rubber articles, such as belts, drive belts and hoses, and/or shoe soles. The mixture compositions known to those skilled in the art for these rubbers, which are specific in terms of fillers, plasticizers, vulcanization systems and additives, are preferably employed.

The natural and/or synthetic polyisoprene of all embodiments may be either cis-1,4-polyisoprene or 3,4-polyisoprene. However, the use of cis-1,4-polyisoprenes having a cis-1,4 proportion of >90% by weight is preferred. Such a polyisoprene is firstly obtainable by stereospecific polymerization in solution with Ziegler-Natta catalysts or using finely divided lithium alkyls. Secondly, natural rubber (NR) is one such cis-1,4-polyisoprene, for which the cis-1,4 content in the natural rubber is greater than 99% by weight.

A mixture of one or more natural polyisoprenes with one or more synthetic polyisoprenes is further also conceivable.

In the context of the present invention the term “natural rubber” is to be understood as meaning naturally occurring rubber which may be obtained from Hevea rubber trees and from “non-Hevea” sources. Non-Hevea sources include for example guayule shrubs and dandelion such as for example TKS (Taraxacum kok-saghyz; Russian dandelion).

If the rubber mixture of the invention contains butadiene rubber (i.e. BR, polybutadiene), this may be any of the types known to those skilled in the art. These include what are called the high-cis and low-cis types, with polybutadiene having a cis content of not less than 90% by weight being referred to as the high-cis type and polybutadiene having a cis content of less than 90% by weight being referred to as the low-cis type. An example of a low-cis polybutadiene is Li—BR (lithium-catalyzed butadiene rubber) having a cis content of 20% to 50% by weight. Particularly good properties and low hysteresis of the rubber mixture are achieved with a high-cis BR.

The polybutadiene(s) employed may be end group-modified with modifications and functionalizations and/or be functionalized along the polymer chains. The modification may be selected from modifications with hydroxyl groups and/or ethoxy groups and/or epoxy groups and/or siloxane groups and/or amino groups and/or aminosiloxane and/or carboxyl groups and/or phthalocyanine groups and/or silane-sulfide groups. However, further modifications known to those skilled in the art, also referred to as functionalizations, are also useful. Metal atoms may be a constituent of such functionalizations.

In the case where at least one styrene-butadiene rubber (styrene-butadiene copolymer) is present in the rubber mixture, this may be selected from solution-polymerized styrene-butadiene rubber (SSBR) and emulsion-polymerized styrene-butadiene rubber (ESBR), a mixture of at least one SSBR and at least one ESBR also being employable. The terms “styrene-butadiene rubber” and “styrene-butadiene copolymer” are used synonymously in the context of the present invention.

The styrene-butadiene copolymer used may be end group-modified and/or functionalized along the polymer chains with the modifications and functionalizations recited above for the polybutadiene.

The at least one diene rubber is preferably selected from the group consisting of natural polyisoprene (NR, natural rubber), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

In a particularly preferred embodiment of the invention the at least one diene rubber is selected from the group consisting of natural polyisoprene (NR), synthetic polyisoprene (IR), butadiene rubber (BR), solution-polymerized styrene-butadiene rubber (SSBR) and emulsion-polymerized styrene-butadiene rubber (ESBR).

In a particularly advantageous embodiment of the invention the rubber mixture comprises at least one natural polyisoprene (NR), preferably in amounts of 50 to 100 phr, and in one particularly advantageous embodiment of the invention 80 to 100 phr, very particularly preferably 95 to 100 phr, in turn preferably 100 phr. Such a rubber mixture especially shows optimized tear properties and abrasion properties coupled with good processability and reversion stability.

If the rubber mixture contains less than 100 phr of NR it preferably contains as a further rubber at least one diene rubber selected from the group consisting of synthetic polyisoprene (IR), butadiene rubber (BR), solution-polymerized styrene-butadiene rubber (SSBR) and emulsion-polymerized styrene-butadiene rubber (ESBR).

In a further particularly advantageous embodiment of the invention the rubber mixture comprises at least one natural polyisoprene (NR), preferably in amounts of 5 to 55 phr, and in one particularly advantageous embodiment of the invention 5 to 25 phr, very particularly preferably 5 to 20 phr. Such a rubber mixture especially exhibits good processability and reversion stability and optimized tear properties and optimal rolling resistance characteristics.

In a further particularly advantageous embodiment of the invention the rubber mixture comprises at least one polybutadiene (BR, butadiene rubber), preferably in amounts of 10 to 80 phr, particularly preferably 10 to 50 phr, and in a particularly advantageous embodiment of the invention 15 to 40 phr. This achieves particularly good tear and abrasion properties of the rubber mixture according to the invention and optimal braking characteristics.

In a further particularly advantageous embodiment of the invention the rubber mixture comprises at least one solution-polymerized styrene-butadiene rubber (SSBR), preferably in amounts of 10 to 80 phr, particularly preferably 30 to 80 phr, and in one particularly advantageous embodiment of the invention 50 to 70 phr. This achieves particularly good rolling resistance properties of the rubber mixture according to the invention. In particularly advantageous embodiments of the invention SSBR is employed in combination with at least one further rubber to achieve an optimal and balanced profile of properties.

It is preferable when the rubber mixture contains at least one filler preferably in amounts of 30 to 500 phr, particularly preferably 50 to 400 phr, in turn preferably 80 to 300 phr.

In advantageous embodiments of the invention the filler is a reinforcing filler which is preferably selected from the group consisting of carbon blacks and silicon dioxide.

Suitable carbon blacks include any carbon black types known to those skilled in the art. It is preferable when the carbon black is selected from industrial carbon blacks and pyrolysis carbon blacks, wherein industrial carbon blacks are more preferred.

It is preferable when the carbon black has an iodine number according to ASTM D 1510, also known as the iodine adsorption number, between 30 and 250 g/kg, preferably 30 to 180 g/kg, particularly preferably 40 to 180 g/kg, and very particularly preferably 40 to 130 g/kg, and a DBP number according to ASTM D 2414 of 30 to 200 ml/100 g, preferably 70 to 200 ml/100 g, particularly preferably 90 to 200 ml/100 g.

The DBP number in accordance with ASTM D 2414 determines the specific absorption volume of a carbon black or a light-colored filler by means of dibutyl phthalate.

The use of such a type of carbon black in the rubber mixture, in particular for vehicle tires, ensures the best possible compromise between abrasion resistance and heat buildup, which in turn influences the ecologically relevant rolling resistance.

A particularly suitable and preferred carbon black is one having an iodine adsorption number between 80 and 110 g/kg and a DBP number of 100 to 130 ml/100 g, such as in particular carbon black of type N339.

The silicon dioxide is preferably amorphous silicon dioxide, for example precipitated silica, which is also referred to as precipitated silicon dioxide. However, it is alternatively also possible to employ pyrogenic silicon dioxide for example.

However, particular preference is given to using a finely divided, precipitated silica which has a nitrogen surface area (BET surface area) (in accordance with DIN ISO 9277 and DIN 66132) of 35 to 400 m²/g, preferably 35 to 350 m²/g, more preferably 85 to 320 m²/g and most preferably 120 to 235 m²/g, and a CTAB surface area (in accordance with ASTM D 3765) of 30 to 400 m²/g, preferably 30 to 330 m²/g, more preferably 80 to 300 m²/g and most preferably 115 to 200 m²/g. Such silicas lead, for example in rubber mixtures for tire treads, to particularly good physical properties of the vulcanizates. Advantages in mixture processing by way of a reduction in mixing time can also result here while retaining the same product properties, leading to improved productivity. Silicas used may thus, for example, be either those of the Ultrasil® VN3 type (trade name) from Evonik or highly dispersible silicas known as HD silicas (e.g. Zeosil® 1165 MP from Solvay).

In particularly advantageous embodiments of the invention the rubber mixture contains at least one silica as filler, preferably in amounts of 30 to 500 phr, particularly preferably 50 to 400 phr, in turn preferably 80 to 300 phr.

In these quantities silica is especially present as the sole or primary filler (more than 50% by weight based on total filler amount).

In further advantageous embodiments of the invention the rubber mixture contains at least one silica as further filler, preferably in amounts of 5 to 100 phr, particularly preferably 5 to 80 phr, in turn preferably 10 to 60 phr.

In these quantities silica is especially present as a further filler in addition to another primary filler, such as in particular a carbon black.

The terms “silicic acid” and “silica” are used synonymously in the context of the present invention.

In particularly advantageous embodiments of the invention the rubber mixture according to the invention contains 0.1 to 60 phr, preferably 3 to 40 phr, particularly preferably 5 to 30 phr, very particularly preferably 5 to 15 phr, of at least one carbon black. In these quantities carbon black is especially present as a further filler in addition to a primary filler, such as in particular silica.

In further advantageous embodiments of the invention the rubber mixture according to the invention contains 30 to 300 phr, preferably 30 to 200 phr, particularly preferably 40 to 100 phr, of at least one carbon black. In these quantities carbon black is present as the sole or primary filler and is optionally present in combination with silica in the abovementioned smaller amounts.

In a particularly advantageous embodiment of the invention the rubber mixture contains 5 to 60 phr, particularly preferably 5 to 40 phr, of at least one carbon black and 50 to 300 phr, preferably 80 to 200 phr, of at least one silica.

The rubber mixture may additionally contain further fillers which are reinforcing or non-reinforcing.

Within the context of the present invention, the further (non-reinforcing) fillers include aluminosilicates, kaolin, chalk, starch, magnesium oxide, titanium dioxide, or rubber gels and also fibers (for example aramid fibers, glass fibers, carbon fibers, cellulose fibers).

Further optionally reinforcing fillers are, for example, carbon nanotubes (CNT), including discrete CNTs, so-called hollow carbon fibres (HCF) and modified CNTs containing one or more functional groups, such as hydroxyl, carboxyl and carbonyl groups), graphite and graphene, and so-called “carbon-silica dual-phase fillers”.

In the context of the present invention zinc oxide is not included among the fillers.

The rubber mixture can further comprise customary additives in customary parts by weight which are added preferably in at least one primary mixing stage during the production of said mixture. These additives include
a) aging stabilizers known in the prior art,
such as for example p-phenylenediamines, such as N-phenyl-N′-(1,3-dimethylbutyl)-p-phenylenediamine (6PPD), N-(1-phenylethyl)-N′-phenyl-p-phenylenediamine (SPPD), N,N′-diphenyl-p-phenylenediamine (DPPD), N,N′-ditolyl-p-phenylenediamine (DTPD), N-(1,4-dimethylpentyl)-N′-phenyl-p-phenylenediamine (7PPD), N-isopropyl-N′-phenyl-p-phenylenediamine (IPPD),
or dihydroquinolines, such as 2,2,4-trimethyl-1,2-dihydroquinoline (TMQ),
b) activators, for example zinc oxide and fatty acids (e.g. stearic acid) and/or other activators, such as zinc complexes, for example zinc ethylhexanoate,
c) activators and/or agents for binding fillers, in particular carbon black or silica, for example S-(3-aminopropyl)thiosulfuric acid and/or metal salts thereof (bonding of carbon black) and silane coupling agents (binding to silicon dioxide, in particular silica),
d) antiozonant waxes,
e) resins, especially tackifying resins,
f) masticating aids, for example 2,2′-dibenzamidodiphenyl disulfide (DBD), and
g) processing aids, such as in particular fatty acid esters and metal soaps, for example zinc soaps and/or calcium soaps,
h) plasticizers, such as in particular aromatic, naphthenic or paraffinic mineral oil plasticizers, for example MES (mild extraction solvate) or RAE (residual aromatic extract) or TDAE (treated distillate aromatic extract), or rubber-to-liquid oils (RTL) or biomass-to-liquid oils (BTL) preferably having a content of polycyclic aromatics of less than 3% by weight according to method IP 346 or triglycerides, for example rapeseed oil or factices or hydrocarbon resins or liquid polymers having a mean molecular weight (determination by GPC=gel permeation chromatography, in accordance with BS ISO 11344:2004) between 500 and 20 000 g/mol.

When using mineral oil this is preferably selected from the group consisting of DAE (distillated aromatic extracts), RAE (residual aromatic extract), TDAE (treated distillated aromatic extracts), MES (mild extracted solvents) and naphthenic oils.

In particularly advantageous embodiments the rubber mixture according to the invention contains no aging stabilizers from the group of p-phenylenediamines, in particular those listed above under a), in addition to the inventive compound of formula I). In a particularly preferred embodiment the rubber mixture according to the invention especially contains 0 to 0.1 phr, in particular 0 phr, of further aging stabilizers based on p-phenylenediamines and selected from the group containing, preferably consisting of, N-phenyl-N′-(1,3-dimethylbutyl)-p-phenylenediamine (6PPD), N-(1-phenylethyl)-N′-phenyl-p-phenylenediamine (SPPD), N,N′-diphenyl-p-phenylenediamine (DPPD), N,N′-ditolyl-p-phenylenediamine (DTPD), N-isopropyl-N′-phenyl-p-phenylenediamine (IPPD), N-(1,4-dimethylpentyl)-N′-phenyl-p-phenylenediamine (7PPD).

The preferably very small amounts of 0 to 0.1 phr, particularly preferably 0 phr, of the recited p-phenylenediamines and the compound of formula I) present according to the invention makes it possible to achieve a comparable protective effect at lower toxicity. The inventive compound of formula I) replaces the recited p-phenylenediamines known in the prior art.

In further advantageous embodiments of the invention at least one further representative of the recited p-phenylenediamine aging stabilizers is present, and so the compound according to the invention only partially replaces the p-phenylenediamines known in the prior art. This also achieves the advantage according to the invention, just not to an optimal extent.

In advantageous embodiments aging stabilizers based on dihydroquinoline, such as TMQ, are present in the rubber mixture in addition to the inventive compound of formula I). The amount of dihydroquinolines present, such as especially TMQ, is preferably 0.1 to 3, in particular 0.5 to 1.5, phr.

Antiozonant waxes (group d above) are considered separately and in preferred embodiments of the invention are present in the rubber mixture irrespective of whether additional aging stabilizers a) are present.

The silane coupling agents may be any of the types known to those skilled in the art.

Furthermore, one or more different silane coupling agents may be used in combination with one another. The rubber mixture may thus contain a mixture of different silanes.

The silane coupling agents react with the surface silanol groups of the silicon dioxide, in particular of the silica, or other polar groups during the mixing of the rubber/the rubber mixture (in situ) or in the context of a pretreatment (premodification) even before addition of the filler to the rubber.

Coupling agents known from the prior art are bifunctional organosilanes having at least one alkoxy, cycloalkoxy or phenoxy group as a leaving group on the silicon atom and having as another functionality a group which, possibly after cleavage, can enter into a chemical reaction with the double bonds of the polymer. The latter group may for example comprise the following chemical groups:
—SCN, —SH, —NH₂ or —Sₓ— (with x=2 to 8).

Employable silane coupling agents thus include for example 3-mercaptopropyltriethoxysilane, 3-thiocyanatopropyltrimethoxysilane or 3,3′-bis(triethoxysilylpropyl) polysulfides having 2 to 8 sulfur atoms, for example 3,3′-bis(triethoxysilylpropyl) tetrasulfide (TESPT), the corresponding disulfide (TESPD), or else mixtures of the sulfides having 1 to 8 sulfur atoms with different contents of the various sulfides. TESPT may for example also be added as a mixture with industrial carbon black (trade name X50S® from Evonik).

Blocked mercaptosilanes as known for example from WO 99/09036 may also be used as a silane coupling agent. It is also possible to use silanes as described in WO 2008/083241 A1, WO 2008/083242 A1, WO 2008/083243 A1 and WO 2008/083244 A1. Employable silanes include for example those marketed by Momentive, USA in a number of variants under the name NXT, such as especially 3-octanoylthio-1-propyltriethoxysilane, or those marketed by Evonik Industries under the name VP Si 363®.

The total proportion of further additives is preferably 3 to 150 phr, more preferably 3 to 100 phr and most preferably 5 to 80 phr.

Zinc oxide (ZnO) may be included in the total proportion of further additives in the abovementioned amounts.

This may be any type of zinc oxide known to those skilled in the art, for example ZnO granules or powder. The zinc oxide conventionally used generally has a BET surface area of less than 10 m²/g. However, it is also possible to use a zinc oxide having a BET surface area of 10 to 100 m²/g, for example so-called “nano zinc oxides”.

The inventive rubber mixture is preferably employed in vulcanized form, in particular in vehicle tires or other vulcanized technical rubber articles.

The terms “vulcanized” and “crosslinked” are used synonymously in the context of the present invention.

The vulcanization of the rubber mixture of the invention is preferably conducted in the presence of sulfur and/or sulfur donors with the aid of vulcanization accelerators, it being possible for some vulcanization accelerators to act simultaneously as sulfur donors. The accelerator is selected from the group consisting of thiazole accelerators, mercapto accelerators, sulfenamide accelerators, thiocarbamate accelerators, thiuram accelerators, thiophosphate accelerators, thiourea accelerators, xanthogenate accelerators and guanidine accelerators.

It is preferable to use a sulfenamide accelerator selected from the group consisting of N-cyclohexyl-2-benzothiazolesulfenamide (CBS), N,N-dicyclohexylbenzothiazole-2-sulfenamide (DCBS), benzothiazyl-2-sulfenomorpholide (MBS), N-tert-butyl-2-benzothiazylsulfenamide (TBBS) and guanidine accelerators such as diphenylguanidine (DPG).

The sulfur donor substances used may be any sulfur donor substances known to those skilled in the art.

Vulcanization retarders may also be present in the rubber mixture.

Production of the rubber mixture according to the invention is preferably otherwise carried out by the process customary in the rubber industry comprising initially producing in one or more mixing stages a primary mixture comprising all constituents except the vulcanization system (for example sulfur and vulcanization-influencing substances). The finished mixture is produced by adding the vulcanization system in a final mixing stage.

The finished mixture is for example processed further and brought into the appropriate shape by means of an extrusion operation or calendering.

The rubber mixture according to the invention is particularly suitable for use in vehicle tires, especially pneumatic vehicle tires. Use in all tire components is conceivable in principle, in particular in an outer component, in particular and preferably in the flange profile, tread and/or sidewall. In the case of a tread with cap/base construction, the rubber mixture according to the invention is preferably used at least in the cap.

For use in vehicle tires the mixture as a finished mixture prior to vulcanization is brought into the corresponding shape, preferably of an outer component, and during production of the green vehicle tire is applied in the known manner.

Production of the rubber mixture according to the invention for use as any other body mixture in vehicle tires is carried out as described above. The difference lies in the shaping after the extrusion operation/the calendering of the mixture. The shapes thus obtained of the as-yet unvulcanized rubber mixture for one or more different body mixtures then serve for the construction of a green tire.

“Body mixture” refers here to the rubber mixtures for the inner components of a tire, such as essentially squeegee, inner liner (inner layer), core profile, belt, shoulder, belt profile, carcass, bead reinforcement, bead profile, flange profile and bandage.

The as-yet unvulcanized green tire is subsequently vulcanized.

For use of the rubber mixture of the invention in drive belts and other belts, especially in conveyor belts, the extruded, as-yet unvulcanized mixture is brought into the appropriate shape and often provided at the same time or subsequently with strength members, for example synthetic fibers or steel cords. This usually affords a multi-ply construction consisting of one and/or more plies of rubber mixture, one and/or more plies of identical and/or different strength members and one and/or more further plies of the same and/or another rubber mixture.

The present invention further provides a vehicle tire comprising the rubber mixture according to the invention containing the compound according to the invention in at least one component.

The vulcanized vehicle tire, in at least one component, comprises a vulcanizate of at least one rubber mixture of the invention. It is known to those skilled in the art that most substances, for example the rubbers present, are present or may be present in chemically modified form either already after mixing or only after vulcanization.

In the context of the present invention “vehicle tires” are to be understood as meaning pneumatic vehicle tires and solid rubber tires, including tires for industrial and construction site vehicles, truck, car and two-wheeled-vehicle tires.

It is preferable when the vehicle tire according to the invention comprises the rubber mixture according to the invention in at least one outer component, wherein the outer component is preferably a tread, a sidewall and/or a flange profile.

The vehicle tire according to the invention may accordingly contain the rubber mixture according to the invention containing the inventive compound of formula I) in a plurality of components, optionally in an adapted composition.

The present invention further provides a process for producing the compound of formula I), wherein the process comprises at least the following process steps:
a1) providing the substance of formula A1) or A2):

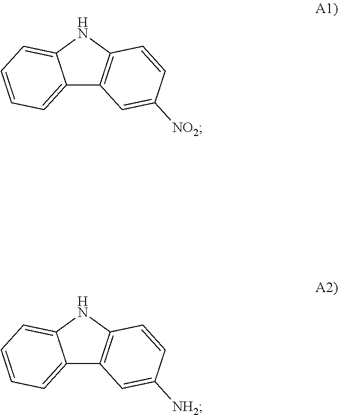 [SMILES: O=[N+]([O-])c1ccc2[nH]c3ccccc3c2c1 ; Nc1ccc2[nH]c3ccccc3c2c1]

b1) providing methyl isobutyl ketone (MIBK) and hydrogen or a reducing agent;
c1) reacting the compound of formula A1) or formula A2) with the substances from step b1) to afford the compound of formula I):

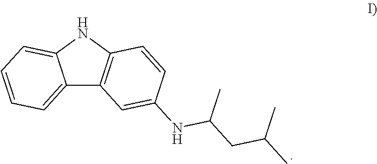 [SMILES: CC(C)CC(C)Nc1ccc2[nH]c3ccccc3c2c1]

The compound of formula A2) may be produced as described in M. Takagi, Org. Biomol. Chem., 2019, 17(7), 1791-1795 and shown in formula R1):

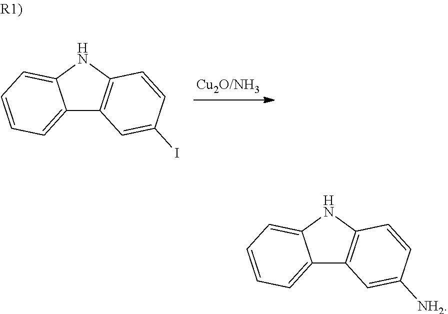 [SMILES: Ic1ccc2[nH]c3ccccc3c2c1 ; Nc1ccc2[nH]c3ccccc3c2c1]

Alternatively, the compound of formula A2) may also be produced by nitration of 9H-carbazole (M. Yu, Supramol. Chem., 2008, 20(4), 357-361) to afford the compound of formula A1) with subsequent reduction (M. Takagi, Org. Biomol. Chem., 2019, 17, 1791-1795) as shown in formulae R2) and R3):

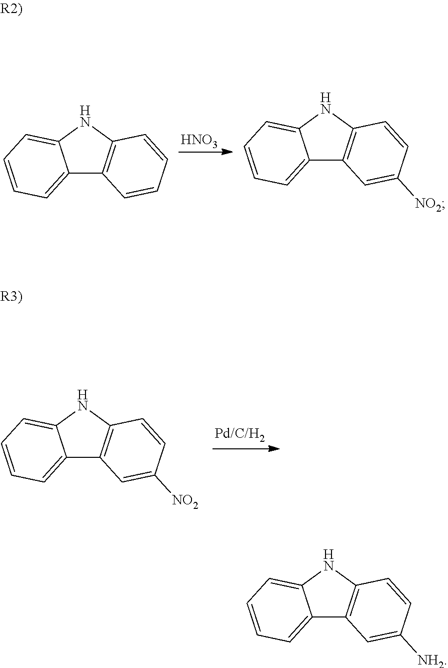

The compound of formula A1) may additionally be produced by condensation of corresponding hydrazines with cyclohexyl ketone and subsequent cyclization/aromatization (B. A. Dalvi, Tet. Lett., 2018, 59, 2145-2149 as shown in formula R4):

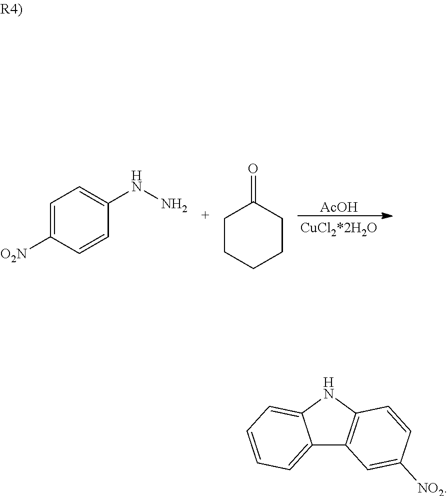 [SMILES: "AcOH" ; NNc1ccc([N+](=O)[O-])cc1 ; O=C1CCCCC1 ; O=[N+]([O-])c1ccc2[nH]c3ccccc3c2c1]

A “reducing agent” is to be understood as meaning a compound which enables a reduction. As is known to those skilled in the art such agents include hydrides, in particular metal hydrides.

A suitable reducing agent is in particular a hydride selected from the group consisting of sodium hydride (NaH), calcium hydride (CaH₂), sodium borohydride (NaBH₄), NaBH₃CN, NaBH(OAc)₃, 2-methylpyridine borane complex.

In the context of the present invention hydrogen is not additionally listed as a “reducing agent” since it is explicitly mentioned as an alternative. It will be appreciated that the term “reducing agent” nevertheless encompasses all reagents that form hydrogen in situ and thus bring about hydrogenation.

The use of a reducing agent—except hydrogen—is especially conceivable when the reaction in step c1) is carried out starting from the compound of formula A2).

The reaction in step c1) is preferably carried out with hydrogen.

The reaction with hydrogen preferably additionally employs a suitable catalyst, referred to in the context of the present invention as “hydrogenation catalyst”.

The hydrogenation catalyst is preferably a noble metal catalyst, such as especially palladium (Pd) or platinum (Pt). The noble metal is preferably employed on carbon (C), such as palladium on carbon (Pd/C).

It is further also possible to employ other known catalysts, such as Raney nickel or copper chromite.

The reaction in step c1) is thus particularly preferably carried out with hydrogen using a hydrogenation catalyst.

The reaction in step c1) is preferably carried out at a temperature of 80° C. to 150° C., especially 120° C. for example.

The reaction mixture is preferably pressurized with hydrogen at a pressure of 35 to 45 bar, in particular 40 bar for example, and then preferably stirred for 1 to 20 hours, preferably 3 to 13 hours, particularly preferably 5 to 13 hours, in particular 10 hours for example.

The reaction with hydrogen in step c1) is preferably carried out in a vessel suitable for the preferably relatively high pressure, such as especially in an autoclave or in another pressure reactor.

The reaction in step c1) is particularly preferably carried out with hydrogen using a hydrogenation catalyst and at a temperature of 80° C. to 150° C. and wherein the reaction mixture is pressurized with hydrogen at a pressure of 25 to 45 bar and the reaction is carried out in an autoclave or in another pressure reactor.

The solvent in step c1) may either be the MIBK which simultaneously serves as a reactant or an inert solvent, such as toluene or xylene. In the latter case MIBK is only employed in stoichiometric amounts as reactant. However, it is preferable when MIBK is simultaneously used as solvent. This makes it possible to avoid an additional substance, such as toluene or xylene. In addition, unconverted MIBK may be recovered and reused following the reaction.

Step c1) is preferably followed by a purification, such as for example by column chromatography, for example over silica gel.

As shown, the reaction in step c1) may be carried out starting either from the compound of formula A1) or the compound of formula A2).

However, in step c1) direct conversion of the compound of formula A1) with hydrogen and MIBK is preferred. This achieves relatively high yields.

The invention shall now be more particularly elucidated below with reference to working examples.

The compound of formula I) was produced as follows according to a first synthetic route, as represented in scheme X1):

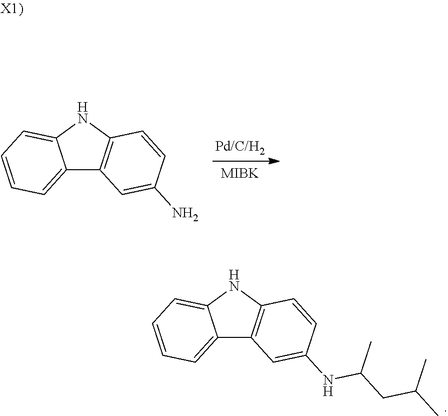 [SMILES: Nc1ccc2[nH]c3ccccc3c2c1 ; CC(C)CC(C)Nc1ccc2[nH]c3ccccc3c2c1]

A stainless steel autoclave provided with a Teflon inliner was charged with 0.35 g (1.92 mmol, 1 eq.) of 3-amino-9H-carbazole (compound of formula A2), 0.16 g of palladium on carbon (Pd/C) (5%) (0.4 g on 4.67 mmol of substrate) and 20.0 ml of methyl isobutyl ketone (MIBK). The reaction mixture was subsequently pressurized with 40 bar of hydrogen (H₂) and stirred for 10 hours at 120° C. After termination of the reaction the excess hydrogen was blown off and the suspension filtered over Celite® with subsequent washing with ethanol. The filtrate was concentrated to dryness and dried under vacuum. The substance was purified over silica gel (cyclohexane/ethyl acetate (EA) 10:1): grayish solid; yield 0.38 g (75% of theory).

¹H-NMR (nuclear magnetic resonance) (500 MHZ, DMSO-d6) δ=10.69 (s, 1H), 7.94 (dd, J=7.7, 1.0 Hz, 1H), 7.36 (d, 8.1 Hz, 1H), 7.27 (ddd, J=8.2, 7.0, 1.2 Hz, 1H), 7.24-7.19 (m, 3H), 7.03 (ddd, J=7.9, 7.0, 1.0 Hz, 1H), 6.78 (dd, J=8.5, 2.3 Hz, 1H), 4.75 (d, J=9.0 Hz, 1H), 3.62-3.47 (m, 1H), 1.80 (dp, J=13.5, 6.7 Hz, 1H), 1.51 (dt, J=13.8, 7.1 Hz, 1H), 1.25 (dt, J=13.5, 6.8 Hz, 2H), 1.13 (d, J=6.1 Hz, 3H), 0.96 (d, J=6.6 Hz, 3H), 0.91 (d, J=6.6 Hz, 3H).

¹³C-NMR (126 MHZ, DMSO-d6) δ=142.3, 140.6, 132.9, 125.2, 123.6, 122.8, 120.3, 117.9, 115.4, 111.9, 111.2, 102.1, 47.0, 46.7, 25.1, 23.4, 23.1, 21.3.

ESI-MS (electrospray ionization mass spectrometry) [M+H]⁺=267.

Melting point: 107° C.

According to a further synthesis route the compound of formula I) was produced as follows as shown in scheme X2):

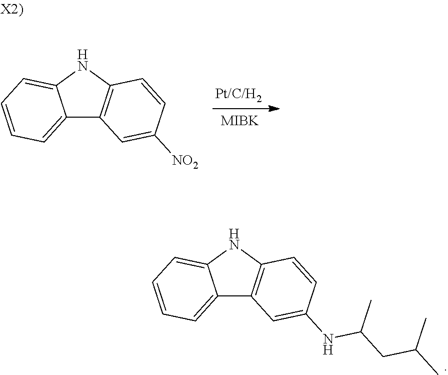 [SMILES: O=[N+]([O-])c1ccc2[nH]c3ccccc3c2c1 ; CC(C)CC(C)Nc1ccc2[nH]c3ccccc3c2c1]

A stainless steel autoclave provided with a Teflon inliner was charged with 4.00 g (18.9 mmol, 1 eq.) of 3-nitro-9H-carbazole (compound of formula A1), 1.60 g of platinum on carbon (Pt/C) (5%) (0.4 g on 4.67 mmol of substrate) and 50.0 ml of methyl isobutyl ketone. The reaction mixture was subsequently pressurized with 40 bar of hydrogen and stirred for 10 hours at 120° C. After termination of the reaction the excess hydrogen was blown off and the suspension filtered over Celite® with subsequent washing with ethanol. The filtrate was concentrated to dryness and dried under vacuum. The substance was purified over silica gel (cyclohexane/EE 10:1): grayish solid; yield 4.40 g (88% of theory).

Analysis of product comprising ¹H-NMR, ¹³C-NMR, ESI-MS and melting point: identical values as for above reaction according to scheme X1).

For use in a rubber mixture for vehicle tires the inventive compound of formula I) is added in one of the mixing stages during production of the rubber mixture in a manner known to those skilled in the art for example instead of the aging stabilizers known in the prior art, such as 6PPD, 7PPD or IPPD etc.

Accordingly the compound of formula I) was incorporated in different amounts into an exemplary rubber mixture according to the invention as shown in table 1. The resulting inventive examples are labelled E1 and E2.

Serving as a comparison are rubber mixtures containing 6PPD instead of the compound of formula I) as aging stabilizer with the remaining composition being identical, V1 and E1 and V2 and E2 undergoing equimolar substitution in each case. The amounts in table 1 are expressed in units of phr. A reference (Ref.) without aging stabilizer is also reported.

In all mixtures the sum of the amounts of aging stabilizer (6PPD or formula I) and plasticizer oil MES is 10 phr.

TABLE 1

| Constituent | V1 | E1 | V2 | E2 | Ref. |
|---|---|---|---|---|---|
| NR | 100 | 100 | 100 | 100 | 100 |
| N 339 | 50 | 50 | 50 | 50 | 50 |
| carbon black | | | | | |
| MES | 8 | 8.01 | 5 | 5.04 | 10 |
| 6PPD | 2 | — | 5 | — | — |
| Compound | — | 1.99 | — | 4.96 | — |
| of formula I) | | | | | |
| ZnO | 3 | 3 | 3 | 3 | 3 |
| Stearic acid | 2 | 2 | 2 | 2 | 2 |
| TBBS | 1.2 | 1.2 | 1.2 | 1.2 | 1.2 |
| Sulfur | 1.2 | 1.2 | 1.2 | 1.2 | 1.2 |

The inventive examples show no significant deterioration or even an improvement in aging stabilization effect relative to rubber mixtures comprising 6PPD.

1. A compound of formula I)

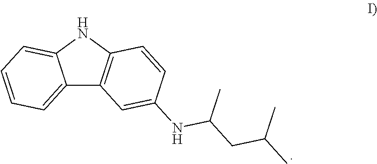 [SMILES: CC(C)CC(C)Nc1ccc2[nH]c3ccccc3c2c1]

2. A rubber mixture containing the compound of formula I) as claimed in claim 1.

3. The rubber mixture as claimed in claim 2, wherein it contains at least one diene rubber.

4. The rubber mixture as claimed in claim 3, wherein it contains at least one diene rubber which is selected from the group consisting of natural polyisoprene (NR), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

5. A vehicle tire which comprises the rubber mixture as claimed in claim 2 in at least one component of the tire.

6. The vehicle tire as claimed in claim 5, wherein in the at least one component is at least one outer component, wherein the outer component is a tread, a sidewall and/or a flange profile.

7. A process for producing the compound of formula I) as claimed in claim 1 comprising at least the following process steps:
a1) providing the substance of formula A1) or A2): 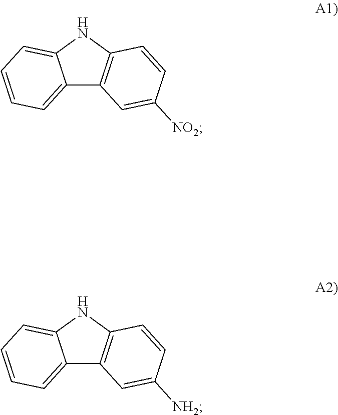 [SMILES: O=[N+]([O-])c1ccc2[nH]c3ccccc3c2c1 ; Nc1ccc2[nH]c3ccccc3c2c1]
b1) providing methyl isobutyl ketone (MIBK) and hydrogen or a reducing agent;
c1) reacting the compound of formula A1) or formula A2) with the substances from step b1) to afford the compound of formula I):

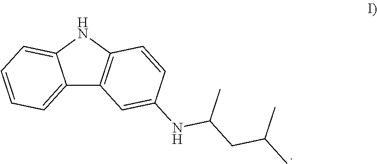 [SMILES: CC(C)CC(C)Nc1ccc2[nH]c3ccccc3c2c1]

8. The process as claimed in claim 7, wherein the reaction in step c1) is carried out with hydrogen using a hydrogenation catalyst and/or at a temperature of 80° C. to 150° C. and/or the reaction mixture is pressurized with hydrogen at a pressure of 25 to 45 bar and the reaction is carried out in an autoclave or in another pressure reactor.

9. A method of using of the compound of formula I) as claimed in claim 1, comprising: (i) providing the compound, and (ii) using the compound as an aging stabilizer and/or antiozonant in vehicle tires and/or other technical rubber articles, including one or more of an air spring, bellows, conveyor belt, belt, drive belt, hose, rubber band, profile, a seal, a membrane, tactile sensors for medical applications or robotics applications, or a shoe sole or parts thereof and/or oils and/or lubricants.

10. A method of using the compound of formula I) as claimed in claim 1, comprising: (i) providing the compound, and (ii) using the compound for producing a rubber article, in particular an air spring, bellows, conveyor belt, belt, drive belt, hose, rubber band, profile, a seal, a membrane, tactile sensors for medical applications or robotics applications, or a shoe sole or parts thereof.

11. A method of using the compound of formula I) as claimed in claim 1, comprising: (i) providing the compound, and (ii) using the compound in oils, lubricants, such as especially fuels or fluids for engines.
